(19) United States
(12) Patent Application Publication
(10) Pub. No.: US 2022/0133953 A1
(43) Pub. Date: May 5, 2022

(54) IMPLANT COMPRISING COATING LAYER FOR OPHTHALMOPATHY

(71) Applicant: SAMSUNG LIFE PUBLIC WELFARE FOUNDATION, Seoul (KR)

(72) Inventors: Chang Won KEE, Seoul (KR); Jong Chul HAN, Seoul (KR); Yan LEE, Seoul (KR); Sun Ah KANG, Seoul (KR); So Hyun PARK, Incheon (KR); In Su BAEK, Gyeonggi-do (KR)

(21) Appl. No.: 17/434,277

(22) PCT Filed: Feb. 5, 2020
(86) PCT No.: PCT/KR2020/001653

§ 371 Date: Aug. 26, 2021

(30) Foreign Application Priority Data
Feb. 27, 2019 (KR) 10-2019-0023214

(51) Int. Cl. A61L 27/34; A61F 9/007

(52) U.S. Cl. CPC A61L 27/34; A61L 2420/06; A61L 2430/16; A61F 9/00781

(57) ABSTRACT

An implant for eye disease for controlling intraocular pressure, the implant including a coating layer formed on the outside of an implant substrate. Specifically, the present invention relates to an implant that may suppress a foreign body reaction from occurring around the implant, because the implant includes a coating layer, which is formed on the outside of an implant substrate and is a cured product of a polymer of a composition containing an acrylate-based monomer and a (meth)acryloyloxyethyl phosphorylcholine monomer.

### TECHNICAL FIELD

This application claims the benefit of the filing date of Korean Patent Application No. 10-2019-0023214, filed with the Korean Intellectual Property Office on Feb. 27, 2019, the entire contents of which are incorporated herein. The present invention relates to an implant for eye disease (ophthalmopathy) for controlling intraocular pressure including a coating layer, and particularly, to an implant for eye disease for controlling intraocular pressure, the implant being capable of preventing fibrosis around the implant.

### BACKGROUND OF THE INVENTION

Glaucoma is a disease in which the optic nerve is pressed or blood supply is obstructed by increased intraocular pressure, causing abnormalities in the function of the optic nerve. The optic nerve is a nerve that transmits the light received by the eye to the brain, so if there is a defect in the optic nerve, a visual field defect appears, and at the end, the sight is lost. The main cause of the onset of glaucoma is damage to the optic nerve due to increased intraocular pressure. The intraocular pressure is mainly determined by aqueous humor (which refers to the liquid water produced in the eye and functions to maintain the shape of the eye and to supply nutrients to the inside of the eye).

Methods for treatment of such glaucoma include a method of instilling or administering an intraocular pressure lowering agent, or glaucoma filtering surgery of making a small hole in the iris by a laser to help the circulation and discharge of aqueous humor. When this drug therapy or the filtering surgery fails or after the drug therapy or filtering surgery is performed, in order to prevent an increase in intraocular pressure, surgery is performed to insert an implant that serves to maintain intraocular pressure at a certain level by controlling the amount of aqueous humor in the eye.

However, even when the surgery is well performed, there is a case in which fibrosis occurs in which the tissue around the implant hardens due to a foreign body reaction around the implant, and eventually intraocular pressure is not controlled again. Thus, in the implant for eye disease, it can be said that it is the most important task to suppress and minimize the foreign body reaction.

Therefore, there is a need for an implant for eye disease that can prevent a foreign body reaction from occurring, that is, prevent in vivo proteins from being adsorbed and denatured thereon, while maintaining the mechanical properties of the implant for eye disease.

### SUMMARY OF THE INVENTION

A technical problem to be achieved by the present invention is to provide an implant for eye disease for controlling intraocular pressure, the implant being capable of preventing a foreign body reaction.

However, the technical problem to be achieved by the present invention is not limited to the above-mentioned problem, and other problems not mentioned herein will be clearly understood by those skilled in the art from the following description.

One embodiment of the present invention provides an implant for eye disease for controlling intraocular pressure, the implant including: an implant substrate; and a coating layer formed on an outside of the implant substrate, wherein the coating layer is a cured product of a polymer of a composition containing: an acrylate-based monomer containing 2 to 6 acrylate groups; and a (meth)acryloyloxyethyl phosphorylcholine monomer.

The implant for eye disease according to one embodiment of the present invention may prevent a foreign body reaction from occurring around the implant.

Specifically, the implant for eye disease according to one embodiment of the present invention may prevent proteins and cells from being adsorbed and denatured on the implant.

### BRIEF DESCRIPTION OF DRAWINGS

FIG. 1 is a schematic view showing a state in which an implant for eye disease for controlling intraocular pressure including a coating layer has been inserted into an eye.

FIG. 2 is a perspective view showing one example of the implant for eye disease for controlling intraocular pressure including a coating layer.

FIG. 3(a)-(c) are sectional views showing one example of the implant for eye disease for controlling intraocular pressure including a coating layer. FIG. 3(a) is a sectional view of the implant for eye disease taken in the X-axis direction of FIG. 2. FIG. 3(b) is a sectional view showing aqueous humor flows into the first regions. FIG. 3(c) is a sectional view showing aqueous humor flow that drains through the second regions.

FIG. 4 is a perspective view showing another example of the implant for eye disease for controlling intraocular pressure including a coating layer.

FIG. 5(a)-(b) are sectional views showing still another example of the implant for eye disease for controlling intraocular pressure including a coating layer. FIG. 5(a) is a sectional view where the rear protective tube of the first tube is formed to surround the outside of the rear tube. FIG. 5(b) is a sectional view where the rear protective tube is formed to be connected to the second region of the first tube, and may be formed to have a thickness larger than the second region of the first tube because it is intended to protect the rear tube from fibrous tissue.

FIG. 6(a)-(b) are perspective views showing an implant for eye disease for controlling intraocular pressure including a coating layer having pores formed therein. FIG. 6(a) is a perspective view showing that the pores for fixing the implant for eye disease may be formed in the second region of the first tube. FIG. 6(b) is a perspective view showing where the rear tube and the rear protection tube are additionally formed, and the pores for fixing the implant for eye disease may be formed in at least one of the second region and the rear protection tube of the first tube.

FIG. 7(a)-(b) depict camera photographs taken 2 weeks after the day of implant insertion. FIG. 7(a) shows the conjunctiva of the right eye into which an implant of Example 1 has been inserted. FIG. 7(b) shows the conjunctiva of the left eye into which an implant of Comparative Example 1 has been inserted.

FIG. 8(a)-(c) schematically show a method of measuring the number of fibroblasts and the thickness of a fibrous capsule in an optical microphotograph of a specimen into which the implant of Example 1 has been inserted. From among 3 conjunctival parts and 3 sclera parts (square parts in FIG. 8(b)) in a part (square part in FIG. 8(a)) where fibrosis was observed, two square parts (square parts in FIG. 8(c)), each having a size of 100×100 μm, were arbitrarily selected, and the number of cells observed in the square was measured.

### DETAILED DESCRIPTION OF THE INVENTION

The terms used in the present invention are currently widely used general terms selected in consideration of their functions in the present invention as possible, but they may change depending on the intention of those skilled in the art, precedent cases, or the emergence of new technology. Additionally, in certain cases, there may be terms arbitrarily selected by the applicant, and in this case, the meaning of the selected terms will be described in detail in the corresponding part of the detailed description of the disclosure. Accordingly, the terms used herein should be defined based on the substantial meaning of the terms and the description throughout the specification, rather than simply the names of the terms.

Throughout the present specification, it is to be understood that when any part is referred to as “including” any component, it does not exclude other components, but may further include other components, unless otherwise specified.

In the present specification, terms such as “ . . . unit”, or “module” mean a unit for processing at least one function or operation, which may be implemented by hardware or software or may be implemented by a combination of hardware and software.

In the present specification, in order to clearly describe the present invention, parts irrelevant to the description in the drawings are omitted. In addition, like reference numerals refer to like parts throughout the specification.

Hereinafter, the present invention will be described in more detail.

One embodiment of the present invention provides an implant for eye disease for controlling intraocular pressure, the implant including: an implant substrate; and a coating layer formed on an outside of the implant substrate, wherein the coating layer is a cured product of a polymer of a composition containing: an acrylate-based monomer containing 2 to 6 acrylate groups and a (meth)acryloyloxyethyl phosphorylcholine monomer.

When the implant according to one embodiment of the present invention is inserted into an eye, the implant may have high biocompatibility, thus preventing a foreign body reaction from occurring around the implant. Specifically, the implant may prevent proteins and cells from being adsorbed and denatured on the implant, thus preventing the occurrence of side effects, for example, fibrosis, thrombus formation, inflammation, or tissue necrosis, in a patient with eye disease.

According to one embodiment of the present invention, examples of the eye disease include glaucoma which is caused by increased intraocular pressure, and example of such glaucoma include congenital glaucoma, traumatic glaucoma, glaucoma suspect, ocular hypertension, primary open-angle glaucoma, normal-tension glaucoma, capsular glaucoma with pseudoexfoliation of lens with pseudo-dropout of the lens, chronic simple glaucoma, low-tension glaucoma, pigmented glaucoma, and primary angle-closure glaucoma, acute angle-closure glaucoma, chronic angle-closure glaucoma, intermittent angle-closure glaucoma, glaucoma secondary to eye trauma, glaucoma secondary to eye inflammation, glaucoma secondary to drug, neovascular glaucoma, or secondary glaucoma caused by uveitis. Therefore, the implant according to one embodiment of the present invention may be an implant for glaucoma.

According to one embodiment of the present invention, the material of the implant substrate may be a material containing functional groups that may provide radicals capable of forming covalent bonds by reaction with the C═C bonds of the acrylate groups of the (meth)acryloyloxyethyl phosphorylcholine monomer contained in the composition. For example, the material of the implant substrate may be at least one selected from among polydimethylsiloxane (PDMS)-based, hydroxyapatite (HA)-based, polylactic acid (PLA)-based, polyglycolic acid (PGA)-based, polytetrafluoroethylene (PTFE)-based, polyethyleneterephthalate (PET)-based, polypropylene-based, polyamide-based, polyacetal-based, polyester-based, and polymethyl methacrylate-based materials. In this case, the coating layer and the material of the implant substrate may form covalent bonds, and thus the coating layer of the implant according to one embodiment of the present invention may have excellent coating strength.

According to one embodiment of the present invention, the composition containing both an acrylate-based monomer containing 2 to 6 acrylate groups and a (meth)acryloyloxyethyl phosphorylcholine monomer is used as a raw material for the coating layer formed on an outside of the implant substrate, whereby it is possible to provide an implant that has excellent coating strength while suppressing a foreign body reaction from occurring around the implant. This is because the acrylate-based monomer containing 2 to 6 acrylate groups acts as a crosslinking agent and is polymerized with the (meth)acryloyloxyethyl phosphorylcholine monomer. That is, the acrylate-based monomer containing 2 to 6 acrylate groups is polymerized while being crosslinked with the (meth)acryloyloxyethyl phosphorylcholine monomer to form a network-like structure. The polymerization may be initiated by radicals formed by a photoinitiator or a thermal initiator. The photoinitiator may be at least one selected from benzophenone, benzoyl peroxide, azobisisobutyronitrile (AIBN), and 2,2-dimethoxy-2-phenylacetophenone (DMPA), but is not limited thereto.

The polymer of the composition containing the acrylate-based monomer and the (meth)acryloyloxyethyl phosphorylcholine monomer may impart hydrophilicity to the coating layer. Accordingly, the coating layer, which is a cured product of the polymer, may have a contact angle with respect to water of 10° to 50°. When the contact angle with respect to water of the coating layer is within the above range, it is possible to prevent proteins and cells from being adsorbed on the implant, thereby preventing the occurrence of side effects, such as fibrosis, thrombus formation, inflammation, and tissue necrosis, in a patient with eye disease. Meanwhile, the coating layer may be a photo-cured or heat-cured product of the polymer.

According to one embodiment of the present invention, the acrylate-based monomer containing 2 to 6 acrylate groups may be at least one selected from among dipentaerythritol pentaacrylate, dipentaerythritol hexaacrylate, ethylene glycol dimethacrylate, and ethylene glycol diacrylate. In this case, the acrylate-based monomer and the (meth)acryloyloxyethyl phosphorylcholine monomer are crosslinked in a network structure, so that the coating layer of the implant according to one embodiment of the present invention may have excellent coating strength.

According to one embodiment of the present invention, the acrylate-based monomer containing 2 to 6 acrylate groups may be contained in an amount of 1 part by weight to 5 parts by weight based on 100 parts by weight of the (meth)acryloyloxyethyl phosphorylcholine monomer. Specifically, the content of the acrylate-based monomer may be 1 part by weight to 4 parts by weight, 1 part by weight to 3 parts by weight, 2 parts by weight to 5 parts by weight, 3 parts by weight to 5 parts by weight, or 2 parts by weight to 4 parts by weight, based on 100 parts by weight of the (meth)acryloyloxyethyl phosphorylcholine monomer. When the content of the acrylate-based monomer is within the above range, the implant according to one embodiment of the present invention may suppress a foreign body reaction from occurring around the implant. In addition, the coating layer of the implant may have excellent strength because crosslinking between the acrylate-based monomer and the (meth)acryloyloxyethyl phosphorylcholine monomer is sufficiently achieved. In addition, the coating layer may be evenly formed on the outside of the implant substrate.

According to an exemplary embodiment of the present invention, the thickness of the coating layer may be 100 nm to 300 nm. Specifically, the thickness of the coating layer may be 100 nm to 250 nm, 150 nm to 300 nm, 150 nm to 250 nm, or 200 nm to 250 nm. When the thickness of the coating layer is within the above range, it is possible to suppress the occurrence of a foreign body reaction (fibrosis) around the implant as well as to minimize foreign body sensation, after insertion of the implant into an eye.

According to one embodiment of the present invention, the implant substrate may include: a first tube into which aqueous humor flows for intraocular pressure control; and a second tube formed inside the first tube, wherein the second tube may be formed of a biodegradable material that is degraded in an eye over a predetermined period of time after being inserted into the eye. In addition, each of the first tube and the second tube may be divided into a first region and a second region according to the distance from the anterior chamber of the eyeball, and the second region of each of the first tube and the second tube may have a larger diameter than that of the first region of each of the first tube and the second tube and may be expandable as the aqueous humor flows thereinto.

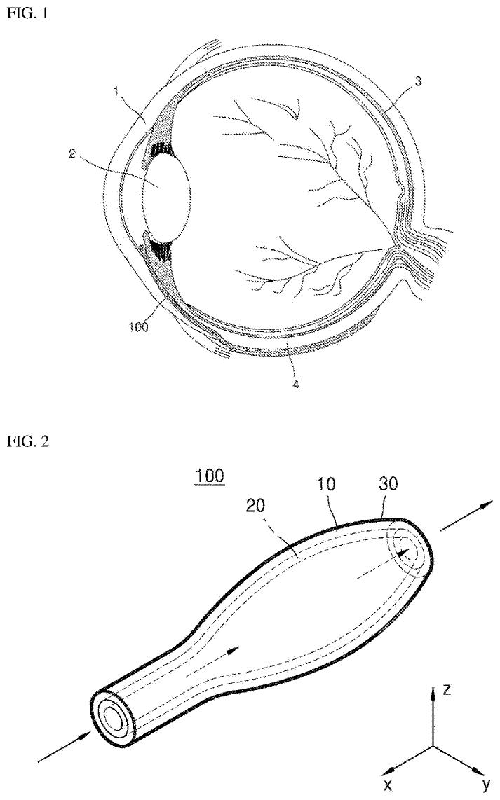

FIG. 1 is a schematic view showing a state in which an implant for eye disease for controlling intraocular pressure including a coating layer according to one embodiment of the present invention has been inserted into an eye.

An implant 100 for eye disease according to one embodiment of the present invention may be inserted after exfoliating the conjunctival tissue or Tenon tissue 4 of the eyeball. After the implant is inserted, it may be disposed in the eye by covering the eyeball again with the conjunctival tissue or Tenon tissue 4. Referring to FIG. 1, when the implant 100 for eye disease is inserted, one side of the implant may be inserted into the anterior chamber of the eyeball and the other side thereof may be inserted into the conjunctival tissue or Tenon tissue 4, so that the aqueous humor produced in the anterior chamber may drain to the conjunctival tissue or Tenon tissue 4 through the implant 100 for eye disease.

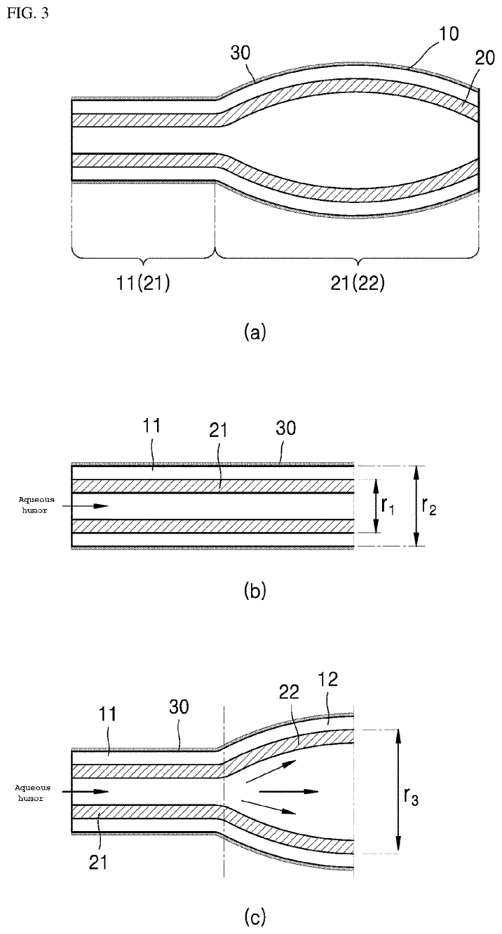

FIG. 2 is a perspective view showing one example of the implant 100 for eye disease for controlling intraocular pressure according to one embodiment of the present invention, and FIG. 3 is a sectional view showing one example of the implant 100 for eye disease for controlling intraocular pressure according to one embodiment of the present invention.

The implant 100 for eye disease according to one embodiment of the present invention may refer to a tube-shaped implant that serves to control intraocular pressure by controlling the drainage of the aqueous humor produced in the anterior chamber of the eyeball in order to prevent the optic nerve from being damaged due to increased intraocular pressure caused by eye disease. Referring to FIGS. 2 and 3, the implant 100 for eye disease for controlling intraocular pressure according to one embodiment of the present invention includes: a first tube 10 into which aqueous humor flows for intraocular pressure control; and a second tube 20 formed inside the first tube 10, wherein a coating layer 30 is formed on an outside of the first tube 10. The second tube 20 may be formed of a biodegradable material that is degraded in an eye over a predetermined period of time after being inserted into the eye. In addition, the first tube 10 and the second tube 20 may be divided into first regions 11 and 21 and second regions 12 and 22 according to the distance from the anterior chamber of the eyeball, and the respective second regions 12 and 22 of the first tube 10 and the second tube 20 may have larger diameters than those of the respective first regions of the first tube 10 and the second tube 20, and may be expandable as the aqueous humor flows thereinto.

The implant 100 for eye disease according to one embodiment of the present invention may include the second tube 20 formed inside the first tube 10 having the coating layer 30 formed on an outside thereof. The second tube 20 may be formed of a biodegradable material that is degraded in the eye over a predetermined period of time. At this time, the biodegradable material may be a material such as collagen or chitin, but is not limited thereto and may include any biodegradable polymer material that is degradable in vivo, causes no various diseases even after degradation, and has no side effects. In addition, the second tube 20 may be formed so that it may be completely degraded preferably within one month, and the second tube 20 may be formed so that the time taken for the second tube to be completely degraded varies depending on the severity of eye disease, the condition of the eyeball. In this case, the implant may control the drainage of aqueous humor over time, and may effectively control intraocular pressure.

In addition, the second region 12 of the first tube 10 may be formed of a biodegradable material that is degraded in an eye over a predetermined period of time, like the above-described second tube 20. That is, the first region 11 of the first tube 10 may be formed of a non-degradable material such as silicone, and the second region 12 thereof may be formed of a biodegradable material such as collagen or chitin, and thus the first region 11 and the second region 12 may be formed of different materials. This is for the purpose of effectively controlling intraocular pressure by controlling the drainage of aqueous humor. The first region 11 of the first tube 10 may be formed of a non-degradable material and thus serve as a channel ensuring that the aqueous humor may continuously drain. When the second region 12 of the first tube 10 is formed of a biodegradable material, like that of the second tube 20, the second regions 12 and 22 are degraded over a predetermined period of time even without separate operation, so that the drainage of aqueous humor may be controlled more easily.

Referring to FIG. 3, regions that are at a relatively close distance from the anterior chamber of the eyeball may be defined as the first regions 11 and 21, and regions, which are connected with the first regions 11 and 21 and are at a relatively far distance from the anterior chamber of the eyeball, may be defined as the second regions 12 and 22. The second regions 12 and 22 are used for the purpose of controlling the pressure of aqueous humor flowing thereinto so that the aqueous humor may drain at an appropriate flow rate and amount. The second region 12 of the first tube 10 may be removed by a method such as opening with a syringe needle by a clinician or the like at a point of time when the second tube 20 is completely degraded, which is a point of time when the above-described purpose is achieved and the second region 12 is no longer necessary. After the second region 22 is removed, only the first region 11 of the first tube 10 remains in the eye so that a predetermined amount of aqueous humor may drain, which makes it possible to control the intraocular pressure.

FIG. 3(a) shows a sectional view of the implant 100 for eye disease taken in the X-axis direction of FIG. 2. That is, referring to FIGS. 2 and 3(a), the respective second regions 12 and 22 of the first tube 10 and the second tube 20 may be formed to have larger diameters than those of the first regions 11 and 21 in the Y-axis direction and may be expandable in the Z-axis direction as aqueous humor flows thereinto. Thus, according to one embodiment of the present invention, the respective first regions 11 and 21 of the first tube 10 and the second tube 20 may be circular in section, and the respective second regions 12 and 22 of the first tube 10 and the second tube 20 may be elliptical in section. In the section of each of the second regions 12 and 22 that may be formed in an elliptical shape as described above, the long diameter may be formed in the Y-axis direction, and the short diameter may be formed in the Z-axis direction.

In addition, in order to allow the second regions 12 and 22 to be easily expandable in the Z-axis direction, the respective second regions 12 and 22 of the first tube 10 and the second tube 20 may be formed to have smaller thicknesses than those of the first regions 11 and 21. The reason why the second regions 12 and 22 each have an expandable structure is to control the pressure that is formed inside the tubes by the inflow of aqueous humor. In order to allow the second regions 12 and 22 to be expanded according to this purpose, the second regions 12 and 22 may be formed to have smaller thicknesses than the first regions 11 and 21 that are formed to have predetermined thicknesses so that aqueous humor may flow therethrough in a fixed state.

According to one embodiment of the present invention, a hole may be formed at the contact point where the first region 11 and the second region 12 of the first tube 10 meet. A medical thread or the like may be connected through the hole, so that the implant 100 for eye disease may be easily fixed into the eye. That is, after the implant 100 for eye disease is inserted into an eye, the implant 100 may be connected, through the hole formed at the contact point where the first region 11 and the second region 12 of the first tube 10 meet, to the tissue in the eye by a medical thread by a clinician or the like, whereby it is possible to prevent the problem that the implant 100 for eye disease moves from the inserted region to another region or shakes due to the pressure of aqueous humor or the fibrous tissue in the eye.

In addition, one surface of the implant 100 for eye disease according to one embodiment of the present invention may be formed in a shape similar to the curved surface of the eyeball. Here, the one surface of the implant 100 for eye disease refers to a portion that is disposed in the direction of the vitreous body of the eyeball when the implant 100 for eye disease is inserted into the eye. When this one surface is formed in a shape similar to the curved surface of the eyeball, the implant 100 may be more stably fixed into the eye.

Referring to FIGS. 3(b) and 3(c), aqueous humor flows into the first regions 11 and 21 and drains through the second regions 12 and 22. The diameters of the second regions 12 and 22 may be larger than those of the first regions 11 and 21 so that the aqueous humor may effectively drain using the pressure of the aqueous humor that flows into the regions. In this case, a diameter r₂ of the first region 11 of the first tube 10 may be 100 μm or more, and a diameter r₁ of the first region 21 of the second tube 20 may be 30 μm to less than 100 μm. Preferably, the diameter r₁ of the first region 21 of the second tube 20 before degradation may be 45 μm. Since the diameters of the second regions 12 and 22 may be larger than the diameters of the first regions 11 and 21 as described above, a diameter r₃ of the second region 22 of the second tube 20 may be larger than the diameter r₁ of the first region 11 of the first tube 10.

In addition, the diameters of the outlets of the respective second regions 12 and 22 of the first tube 10 and the second tube 20 through which aqueous humor drains, may be equal to or larger than those of the inlets of the first regions 11 and 21 of the first tube 10 and the second tube 20 into which aqueous humor flows. In other words, since the diameters of the outlets finally determine the flow rate of aqueous humor that drains through the implant 100 for eye disease, the outlets of the respective second regions 12 and 22 of the first tube 10 and the second tube 20 may be selectively formed to have diameters equal to or larger than those of the inlets of the first regions 11 and 21, so that the drainage rate and drainage amount of the aqueous humor are controlled according to the severity of eye disease, the condition of the eyeball, etc.

According to one embodiment of the present invention, the lengths of the respective first regions 11 and 21 of the first tube 10 and the second tube 20 may each be 7 mm to 10 mm. That is, as described above, after the second region 12 is removed by a clinician, etc., aqueous humor must drain from the anterior chamber of the eyeball to the conjunctival tissue or Tenon tissue through only the first region 11. Thus, each of the first regions 11 and 21 may be formed to have a length within the above range so that the aqueous humor stably drains.

A clinically remarkable effect (e.g., proper maintenance of aqueous humor or drainage of aqueous humor) may appear within the above-described numerical ranges, but the scope of the present invention is not necessarily limited to these values. The implant may be manufactured to have a diameter or length value predetermined depending on the patient's eyeball size, treatment timing, period, and the like.

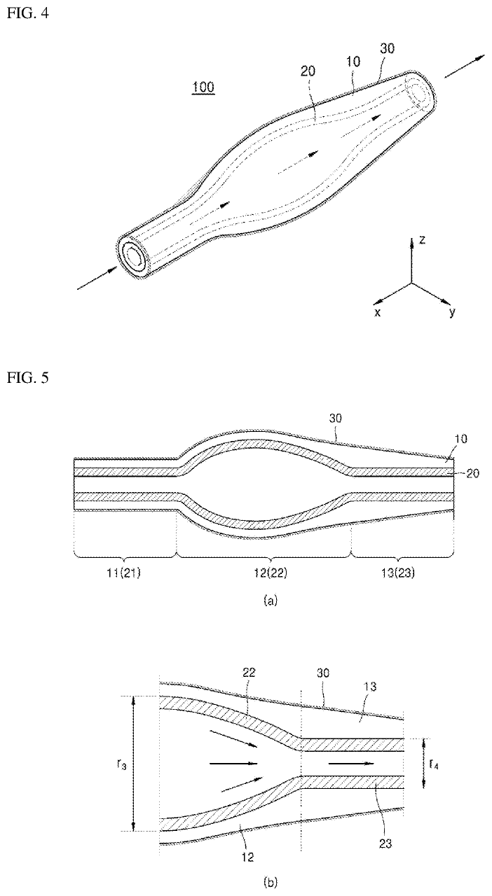

FIG. 4 is a perspective view showing another example of the implant 100 for eye disease for controlling intraocular pressure according to one embodiment of the present invention, and FIG. 5 is a sectional view showing still another example of the implant 100 for eye disease for controlling intraocular pressure according to one embodiment of the present invention.

Referring to FIGS. 4 and 5, the second tube 20 according to one embodiment of the present invention further includes a rear tube 23 connected to the second region 22 of the second tube 20 and formed to have a diameter equal to or smaller than the first region 21 of the second tube 20, and the first tube 10 may further include a rear protective tube 13 connected to the second region 12 of the first tube 10 and configured to surround the outside of the rear tube 33 so as to protect the rear tube 23.

In other words, referring to FIG. 5, the rear tube 23 of the second tube 20 may be additionally formed to be connected with the second region 22 of the second tube 20 for effective control of the pressure of aqueous humor. A diameter r₄ of the rear tube 23 may be equal to or smaller than the diameter r₁ of the first region 21 of the second tube 20 depending on the drainage amount and drainage rate of aqueous humor determined according to the severity of eye disease, the condition of the eyeball, etc.

In addition, referring to FIG. 5(a), the rear protective tube 13 of the first tube 10 may be formed to surround the outside of the rear tube 23 so as to prevent the rear tube 23 of the second tube 20 from being clogged or damaged by fibrous tissue. The rear protective tube 13 may be formed to be connected to the second region 12 of the first tube 10, and may be formed to have a thickness larger than the second region 12 of the first tube 10 because it is intended to protect the rear tube 23 from fibrous tissue. In addition, the rear protective tube 13 and the rear tube 23 may be removed by a method such as opening with a syringe needle by a clinician or the like at a point of time when the second tube 20 is completely degraded, like the respective second regions 12 and 22 of the first tube 10 and the second tube 20. In addition to the above-described method, when the rear protective tube and the rear tube 23 are formed of a biodegradable material as described above, they are naturally degraded over a predetermined period of time, so that the pressure and drainage amount of aqueous humor may be easily controlled.

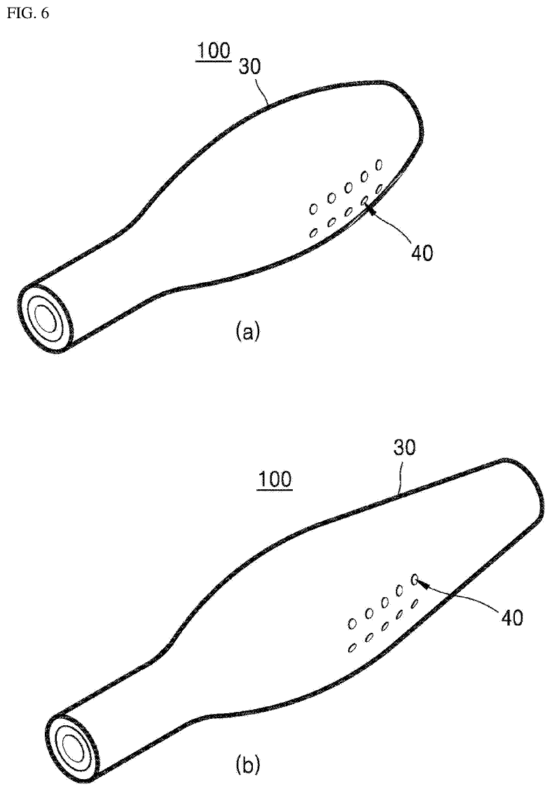

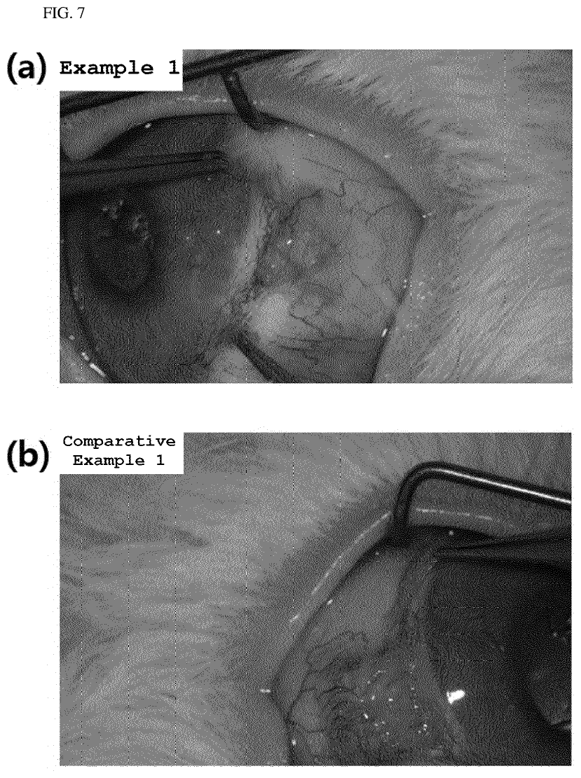

FIG. 6 is a perspective view showing an implant 100 for eye disease for controlling intraocular pressure including pores 40 formed therein according to one embodiment of the present invention.

Referring to FIG. 6(a), the pores 40 for fixing the implant 100 for eye disease may be formed in the second region 12 of the first tube 10. That is, when the pores 40 are formed in the second region 12 of the first tube 10, fibrous tissue of the eyeball grown over time after the insertion of the implant 100 for eye disease into the eye may enter the pores, thus fixing the implant 100 for eye disease. When the pores 40 are formed in the second tube 20, the fibrous tissue may enter the tube and interfere with the movement of aqueous humor. For this reason, the pores 40 may be preferably formed in the first tube 10, but may also be formed in the second tube 20 if necessary.

In addition, as shown in FIG. 6(b), even when the rear tube 23 and the rear protection tube 13 are additionally formed, the pores 40 for fixing the implant 100 for eye disease may be formed in at least one of the second region 12 and the rear protection tube 13 of the first tube 10. As described above, the rear protective tube 13 is formed to have a larger thickness than the second region 12 of the first tube 10 so as to protect the rear tube 23. Thus, the pores 40 are preferably formed in the rear protective tube 13, but the scope of the present invention is not limited thereto.

Hereinafter, the present invention will be described in detail with reference to examples. However, the examples according to the present invention may be modified into various different forms, and the scope of the present invention is not interpreted as being limited to the examples described below. The examples of the present specification are provided to more completely explain the present invention to those skilled in the art.

### Example 1

An implant substrate (formed of polydimethylsiloxane or polytetrafluoroethylene) having a size of 3*3*0.4 mm was prepared.

A composition was prepared which contains 1 part by weight of dipentaerythritol pentaacrylate and 2 parts by weight of ethylene glycol dimethacrylate as acrylate-based monomers, 100 parts by weight of a methacryloyloxyethyl phosphorylcholine monomer, and benzophenone as a photoinitiator.

The composition was applied onto the implant substrate and irradiated with UV light, thereby producing an implant for eye disease for controlling intraocular pressure, which has formed therein a coating layer which is a photocured product of the photopolymer of the composition.

### Comparative Example 1

As an implant, an implant substrate (formed of polydimethylsiloxane or polytetrafluoroethylene) having a size of 3*3*0.4 mm and having no coating layer was prepared.

Production of Experimental Animal Model and Observation of Conjunctiva

The implant of Example 1 was inserted into the right eye of each of 12 New Zealand white rabbits having a weight of 1.5 kg to 2.0 kg, and the implant of Comparative Example 1 was inserted into the left eye of each rabbit.

The conjunctiva of each of the right eye into which the implant of Example 1 has been inserted and the left eye into which the implant of Comparative Example 1 has been inserted was visually observed 2 weeks after the date of implant insertion. FIG. 7 depicts camera photographs taken 2 weeks after the date of implant insertion, which show the conjunctiva of each of the right eye into which the implant of Example 1 has been inserted and the left eye into which the implant of Comparative Example 1 has been inserted.

Measurement of Number of Fibroblasts and Thickness of Fibrous Capsule

2 weeks after insertion of the implants, the 12 rabbits were anesthetized by injection of 8 ml/kg of a 1:1 mixture of Tiletamine/Zolazepham, and the eyeballs were extracted. During eyeball extraction, the conjunctiva and Tenon tissue around the limbus were exfoliated to be included in the eyeballs so that they did not fall off. Then, the eyeballs were immersed and fixed in 4% paraformaldehyde diluted with 0.4 M phosphate buffer (PB; pH 7.4) for 3 hours. The eyeballs were taken out of the fixative, divided into axial sections using a razor blade, and then immersed in and washed with 0.1 M PB. The eyeballs were dehydrated stepwise with ethanol, and then covered with wax (polyethylene glycol 400 distearate; Polysciences, Warrington, Pa.). The eyeballs were was cut into sagittal sections of 5 μm in size using a microtome, and then placed on gelatinized slide, thus preparing specimens. After the wax was removed, the specimens were subjected to rehydration and H & E (hematoxylin & eosin) staining.

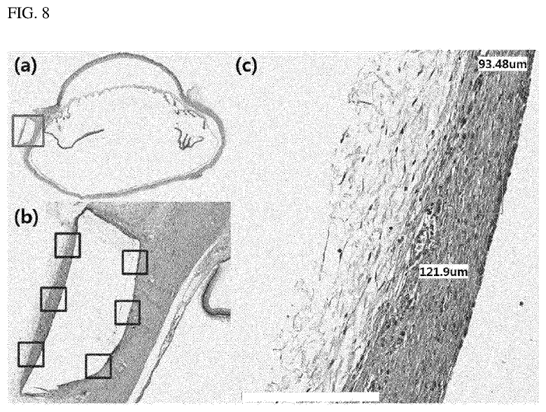

The number of fibroblasts and the thickness of a fibrous capsule in the Tenon tissue were measured using an optical microscope at 200× magnification. FIG. 8 schematically shows a method of measuring the number of fibroblasts and the thickness of the fibrous capsule in an optical microphotograph of the specimen into which the implant of Example 1 has been inserted. Specifically, from among 3 conjunctival parts and 3 sclera parts (square parts in FIG. 8(b)) in a part (square part in FIG. 8(a)) where fibrosis was observed, two square parts (square parts in FIG. 8(c)), each having a size of 100×100 μm, were arbitrarily selected, and the number of cells observed in the square was measured. In addition, the thickness of the fibrous capsule at any point near the square was measured. Table 1 below shows the respective average values of the numbers of cells and the thicknesses of the fibrous capsules in 3 conjunctiva parts and 3 sclera parts.

TABLE 1

| | Average value of | Average value (μm) of |
|---|---|---|
| | numbers of | thicknesses of |
| | fibroblasts | fibrous capsules |
| Example 1 | 17.7 | 130.7 |
| Comparative | 33.8 | 263.3 |
| Example 1 | | |

Referring to FIG. 7, it could be confirmed that the degree of redness decreased in the right eye into which the implant of Example 1 has been inserted, compared to the left eye into which the implant of Comparative Example 1 has been inserted. In addition, referring to Table 1 above, it could be confirmed that the number of fibroblasts and the thickness of the fibrous capsule around the implant in the right eye into which the implant of Example 1 has been inserted were smaller than the number of fibroblasts and the thickness of the fibrous capsule around the implant in the right eye into which the implant of Comparative Example 1 has been inserted.

Therefore, it could be seen that the implant for eye disease according to one embodiment of the present invention may include, on the outside of the implant substrate, the coating layer which is a cured product of a polymer of a composition containing: an acrylate-based monomer containing 2 to 6 acrylate groups; and a (meth)acryloyloxyethyl phosphorylcholine monomer, and thus prevent cells and proteins from being adsorbed and denatured thereon, thus preventing foreign body reactions such as fibrosis, thrombus formation, inflammation, or tissue necrosis.

### DESCRIPTION OF REFERENCE NUMERALS

| | |
|---|---|
| 1: eye's cornea; | 2: eye's lens; |
| 3: eye's retina; | 4: eye's conjunctival tissue or |
| 10: first tube; | Tenon tissue; |
| 12: second region | 11: first region of first tube; |
| of first tube; | 13: rear protective |
| 20: second tube; | tube of first tube; |
| 22: second region | 21: first region of second tube; |
| of second tube; | 23: rear tube |
| 30: coating layer; | of second tube |
| 100: implant for eye disease | 40: pores |

1. An implant for eye disease for controlling intraocular pressure, the implant comprising:
an implant substrate; and
a coating layer formed on an outside of the implant substrate,
wherein the coating layer is a cured product of a polymer of a composition containing: an acrylate-based monomer containing 2 to 6 acrylate groups; and a (meth)acryloyloxyethyl phosphorylcholine monomer.

2. The implant of claim 1, wherein the coating layer has a contact angle with respect to water of 10° to 50°.

3. The implant of claim 1, wherein the acrylate-based monomer is at least one selected from among dipentaerythritol pentaacrylate, dipentaerythritol hexaacrylate, ethylene glycol dimethacrylate, and ethylene glycol diacrylate.

4. The implant of claim 1, wherein the acrylate-based monomer is contained in an amount of 1 parts by weight to 5 parts by weight based on 100 parts by weight of the (meth)acryloyloxyethyl phosphorylcholine monomer.

5. The implant of claim 1, wherein the coating layer has a thickness of 100 nm to 300 nm.

6. The implant of claim 1, wherein the implant substrate comprises:
a first tube into which aqueous humor flows for intraocular pressure control; and
a second tube formed inside the first tube,
wherein the second tube is formed of a biodegradable material that is degraded in an eye over a predetermined period of time after being inserted into the eye,
each of the first tube and the second tube is divided into a first region and a second region according to the distance from an anterior chamber of the eye, and
the second region of each of the first tube and the second tube has a larger diameter than that of the first region of each of the first tube and the second tube and is expandable as aqueous humor flows thereinto.

7. The implant of claim 6, wherein the first region of each of the first tube and the second tube is circular in section, and the second region of each of the first tube and the second tube is elliptical in section.

8. The implant of claim 6, wherein the first region of the first tube has a diameter of 100 μm or more, and the first region of the second tube has a diameter of 30 μm or more and less than 100 μm.

9. The implant of claim 6, wherein the first region of each of the first tube and the second tube has a length of 7 mm to 10 mm.

10. The implant of claim 6, wherein the second tube further comprises a rear tube connected to the second region of the second tube and formed to have a diameter equal to or smaller than the first region of the second tube, and
the first tube further comprises a rear protective tube connected to the second region of the first tube and configured to surround an outside of the rear tube so as to protect the rear tube.
